(19) United States
(12) Patent Application Publication
(10) Pub. No.: US 2008/0149117 A1
(43) Pub. Date: Jun. 26, 2008

(54) Method of increasing medical hygiene using color coded medical devices

(76) Inventor: Akhil Raghuram, Vancouver, WA (US)

Correspondence Address: KURT M. RYLANDER;RYLANDER & ASSOCIATES PC, 406 West 12th Street, Vancouver, WA, 98660, US

(21) Appl. No.: 11/644,401

(22) Filed: Dec. 21, 2006

(51) Int. Cl. A61B 19/00

(52) U.S. Cl. 128/898

(57) ABSTRACT

A method of increasing medical hygiene, includes providing differently colored medical devices, providing one or more different patient environments, assigning each patient environment a unique color chosen from the colors represented by the differently colored medical devices, placing medical devices in patient environments having the same color assignment as the color of the medical device, identifying and removing medical devices from a patient environment where the devices in the patient environment do not match the assigned color of the patient environment. The medical aid can be protective medical clothing, such as surgical gloves.

### FIELD OF THE INVENTION

The present invention relates to color coded surgical gloves. More particularly, the present invention relates to color coded surgical gloves for hospital room hygiene.

### BACKGROUND

The need for cleanliness and sterilization in the practice of medicine is paramount and is one of the fundamental requirements for a successful medical practice. This is true for a large hospital with multiple surgery rooms as well as a small family practitioner.

The use of different types of sterile gloves, aprons, hairnets, masks and other types of clothing used to prevent the spread or cross contamination of particles harmful to the wellbeing of the patient, the health of the doctor and the successful practice of medicine, are employed for the purpose of maintaining a sterile environment for which to practice medicine. By employing these protective articles of clothing, the risk of contamination from doctor to patient and vice versa is reduced. The most common method of ensuring that the practice of “donning” new gloves whenever the doctor changes rooms or works with a new patient is to remove the gloves as soon as the task for which they were used in the first place is complete, or when the doctor leaves the room. If the doctor forgets to remove the gloves after finishing the task it is possible that the same gloves could be used to perform a task involving another patient.

Thus, there is a need for a quick method of ensuring that gloves are always changed when entering a new room or environment, that sterile gloves are always used when performing a new task and that will reduce the risk of reusing the same gloves for multiple tasks or patients.

A number of devices have provided for the color coding for identification purposes of surgical gloves, medical instruments and other devices where a desirable characteristic is the ability to quickly differentiate between either sizes or models, but lack the teaching of a method for segregating devices by environment or specific hospital location such as a room or ward for the purpose of preventing cross contamination between patients, staff and location. The following represents a list of known related art:

| | | Date of |
|---|---|---|
| | | Issue/ |
| Reference: | Issued to: | Publication: |
| U.S. Pat. App. Pub.No. 2005/0170726 | Brunson et. al. | Aug. 4, 2005 |
| U.S. Pat. App. Pub.No. 2003/0047126 | Tomaschko | Mar. 13, 2003 |
| U.S. Pat. App. Pub.No. 2002/0010957 | Katz | Jan. 31, 2002 |
| U.S. Pat. No. 6,223,440 | Rashman | May 1, 2001 |
| U.S. Pat. No. 5,573,529 | Haak et. al. | Nov. 12, 1996 |
| U.S. Pat. No. 5,442,816 | Seketa | Aug. 22, 1995 |
| U.S. Pat. No. 5,275,612 | Bales, Jr. | Jan. 4, 1994 |
| U.S. Pat. No. 5,231,700 | Cutshall | Aug. 3, 1993 |
| U.S. Pat. No. 5,173,966 | DeLeo | Dec. 29, 1992 |
| U.S. Pat. No. 4,671,916 | Hamas | Jun. 9, 1987 |
| U.S. Pat. No. 3,994,643 | Joslin et. al. | Nov. 30, 1976 |
| U.S. Pat. No. 3,975,776 | Ayres | Aug. 24, 1976 |
| U.S. Pat. No. 3,852,826 | Schindler | Dec. 10, 1974 |

The teachings of each of the above-listed citations (which does not itself incorporate essential material by reference) are herein incorporated by reference. None of the above inventions and patents, taken either singularly or in combination, is seen to describe the instant invention as claimed.

U.S. patent application 20030047126 A1 to Tomaschko teaches a method for identifying medical devices by manufacturing the device with either a distinguishing characteristic or a distinguishing color. Tomaschko does not teach a method for employing these distinguishing features or colors to prevent the cross contamination from one environment to another.

U.S. Pat. No. 6,223,440 to Rashman teaches a method for marking autoclavable instruments for the purpose of segregating them by hospital ward. Rashman discloses this as a method to reduce transmission of infectious disease between hospital wards and as a method to reduce the risk of loss and theft for the autoclavable instruments. Rashman does not teach a method for increasing the sterility and risk of cross contamination between medical environments by the use of colored surgical gloves nor does Rashman teach a method for carrying out this segregation. Rashman merely teaches a method for marking autoclavable devices and marking them with a color.

U.S. Pat. No. 5,573,529 to Haak teaches a method for marking metal medical instruments by color for the purpose of identification. Haak does not teach a method for using color coded instruments to increase medical hygiene.

U.S. Pat. No. 4,671,916 to Hamas teaches a method of marking instruments to indicate they belong to a set and facilitate identification of whether the set is complete. Hamas does not teach a method for employing said marking system and specifically Hamas does not teach a method for using said system to increase medical hygiene.

Thus, while the foregoing body of art indicates it to be well known to mark medical instruments by color for the purpose of identification, the art described above does not teach or suggest a method for employing said marking system which has the following combination of desirable features: (1) increased surety that medical devices such as gloves are not removed from specific medical or patient environments; (2) decreasing the risk of cross contamination, and increasing the sterility of the medical or patient environment.

### SUMMARY AND ADVANTAGES

A method of increasing medical hygiene is provided which includes providing differently colored medical devices, providing one or more different patient environments, assigning each patient environment a unique color chosen from the colors represented by the differently colored medical devices, placing medical devices in patient environments having the same color assignment as the color of the medical device, identifying and removing medical devices from a patient environment where the devices in the patient environment do not match the assigned color of the patient environment. The medical aid can be protective medical clothing, such as surgical gloves.

The method of the present invention presents numerous advantages, including: (1) the ability for the medical practitioner to quickly and easily identify if a medical instrument or device such as a glove belongs in that medical environment; (2) the ability for any person to determine quickly and easily if the medical instrument or device belongs in that medical environment, thereby removing the need for a single person to remember if they have changed their gloves or carried a medical device from one location to another, since the distinguishing factor is determinable by anyone. Additional advantages of the invention will be set forth in part in the description which follows, and in part will be obvious from the description, or may be learned by practice of the invention. The advantages of the invention may be realized and attained by means of the instrumentalities and combinations particularly pointed out in the appended claims. Further benefits and advantages of the embodiments of the invention will become apparent from consideration of the following detailed description given with reference to the accompanying drawings, which specify and show preferred embodiments of the present invention.

### BRIEF DESCRIPTION OF THE DRAWINGS

The accompanying drawing, which is incorporated into and constitute a part of this specification, illustrate one or more embodiments of the present invention and, together with the detailed description, serve to explain the principles and implementations of the invention.

FIG. 1 shows a flow chart detailing the steps involved in practicing this invention.

### DETAILED DESCRIPTION

Before beginning a detailed description of the subject invention, mention of the following is in order. When appropriate, like reference materials and characters are used to designate identical, corresponding, or similar components in differing figure drawings. The figure drawings associated with this disclosure typically are not drawn with dimensional accuracy to scale, i.e., such drawings have been drafted with a focus on clarity of viewing and understanding rather than dimensional accuracy.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

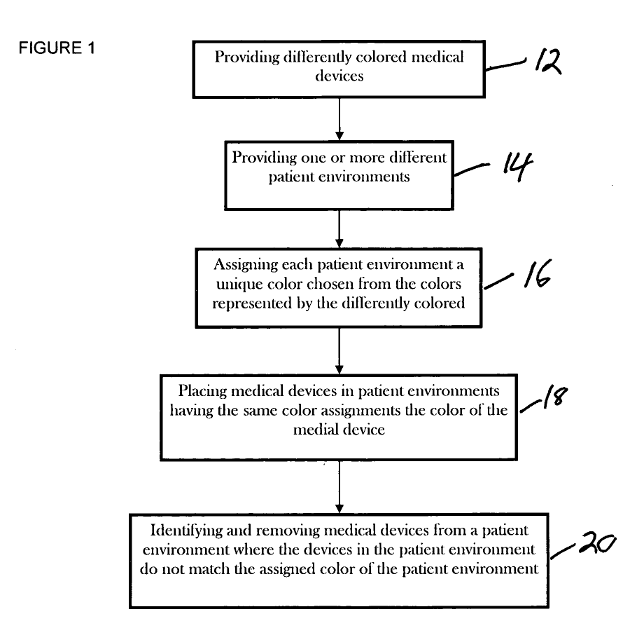

As shown in the flow chart in FIG. 1, a method of increasing medical hygiene 10, includes providing differently colored medical devices 12, providing one or more different patient environments 14, assigning each patient environment a unique color chosen from the colors represented by the differently colored medical devices 16, placing medical devices in patient environments having the same color assignment as the color of the medical device 18, identifying and removing medical devices from a patient environment where the devices in the patient environment do not match the assigned color of the patient environment 20. The medical aid can be protective medical clothing, such as surgical gloves.

The method for using different colored surgical gloves in different medical environments includes assigning each patient environment such a different color or other identifying characteristic such that when the medical provider enters the room, it will be a simple task to ensure than medical devices such as gloves which do not have said room characteristic are not used in said room.

A method of increasing medical hygiene, includes providing a plurality of patient environments each having a different assigned color, providing a plurality of surgical gloves each having an assigned color chosen from the assigned colors used for patient environments, placing surgical gloves in patient environments by matching surgical glove color to patient environment color, removing from a patient environment any surgical glove which does not match the assigned color.

A method for maintaining cleanliness in a medical environment includes assigning different environments within the medical facility, where it is desired to either maintain cleanliness or prevent the spread of possible contaminants into or out of the environment, with a specific color providing sterilized gloves or other desired devices with the corresponding color in each designated environment, and employing a procedure which requires identifying and removing items which are not of the designated color when worn or carried into the environment.

The method can be practiced by designating different environments within the medical facility and assigning them individual colors, then placing within each designated environment surgical gloves or other desired protective clothing such as a mask or hair net that corresponds with the specific environments assigned color. For example a small practitioner's office with two patient exam rooms, Room A and Room B. Room A could be assigned color X and Room B assigned color Y. If the practitioner were to enter Room A with gloves of color Y, it would be obvious to any observer who knows the color scheme that the sterility of the office may be compromised and the practitioner's gloves need to be changed. This same type of system can be applied to any device or clothing item which it is desired to keep segregated or quarantined to specific environments.

In the preferred embodiment, there are two or more patient examination rooms on a hospital floor. In each room, a box of surgical gloves is provided for use by the medical personnel. Each patient examination room is assigned a specific color, preferably chosen from red, yellow, blue, pink, green, and brown. The surgical gloves in each surgical room match the assigned color—for example, pink surgical gloves in a pink assigned examination room, red surgical gloves in a red assigned examination room, yellow surgical gloves in a yellow assigned examination room and so forth. When a medical provider is in an examination room and has on surgical gloves which do not match the assigned room color (determined by looking at the room assignment, which can be determined for example by simply looking at the box of surgical gloves already in the room), the medical provider knows the surgical glove is from another examination room and immediately disposes of it and dons a correctly colored surgical glove from the box provided in the current examination room.

Those skilled in the art will recognize that numerous modifications and changes may be made to the preferred embodiment without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the preferred embodiment is essential. Other embodiments are possible, their specific designs depending upon the particular application. As such, the scope of the invention should not be limited by the particular embodiments herein described but should be defined only by the appended claims and equivalents thereof.

I claim:

1. A method of increasing medical hygiene, comprising:
providing differently colored medical devices;
providing one or more different patient environments;
assigning each patient environment a unique color chosen from the colors represented by the differently colored medical devices;
placing medical devices in patient environments having the same color assignment as the color of the medical device;
identifying and removing medical devices from a patient environment where the devices in the patient environment do not match the assigned color of the patient environment.

2. The method claim of claim 1 wherein said medical device is protective medical clothing.

3. The method claim of claim 1 wherein said medical device is a surgical glove.

4. A method for using different colored surgical gloves in different medical environments includes assigning each patient environment such a different color or other identifying characteristic such that when the medical provider enters the room, the provider can visually determine that medical devices which do not have said room characteristic are not used in said room.

5. A method of increasing medical hygiene, comprising steps of:
providing a plurality of patient environments each having a different assigned color;
providing a plurality of surgical gloves each having an assigned color chosen from the assigned colors used for patient environments;
placing surgical gloves in patient environments by matching surgical glove color to patient environment color; and
removing from a patient environment any surgical glove which does not match the assigned color.
